SEQUENCE LISTING (7 sequences)

SEQ ID NO: 1 - DNA, 352 bp, synthetic construct
  source 1..352: mol_type = other DNA
tatttttcattcgaaaaaaatagaggggttactggcaaatatattgttggtaaccctataattatgtttttattaatatggaatatatgggaaaaggaaaataatgaccaaaaaacgaacattaatcaaaaaatgaacaccattttttgattactttgatctaaaaatgaacacaacataataagcatcataaaaacactacctaatttggacactttcaacattggcatacatattgcttatataaatagtaacattaaatatttcgaccaacattaaaacgcatctacaatttgtaatatgaatgcgatttaaattgcaagttgtagataaaaaattgaggaggtatagt

SEQ ID NO: 2 - DNA, 987 bp, synthetic construct
  source 1..987: mol_type = other DNA
agtttctaagctaaaaataaacttatatagagttcctaaacaaaaaatcaccttatattccggatattttttacaacattcagctcggttaaatattttgataagcctaagtaaaaaattctagaaaatctaagaacttttgaaaactcgtacctcaaacaattcaaagttctaagtttttacgaattttttactaagatttatttgcaaaatatttaaatgagcttcaccgttgtaaaaatatgcctacatataaggtgatttttaagttagaaagtgtggcataggtttatttttttagttataacacttgtatatttaattctacatagagtggagctgaagattgtaaaaatatctggagaataggttaatttctaacttagaagtctttcattaattccgtttatttttccactgggcgataagtacatatcctaaaaatataaggactaatcctataatcatagtaaaaaaagtttttataacattttcccaatttgatgcgttaaaagcacccatagtccagaatacaatagtaggaagttgctcataggggtcagctatgtattttaaaaaagaaagtcctgcaggaaaaagtgaagaaatgattattacagctaacactaatgtagttcaatatcctttaaattttgagcaacattaatgcaaaataaatgtaaataaatgatgatatttaaaatgcgttcgaaaaacgaactatagtcaaaaaacgaacacagcttttttaattgatttgatcaaaaagtgaacatacataatagaaaatataaaagaaactacctaatttgggcattttcaacaatggcatatatattgcttataagtaaataatgactctaaatttaaacatttaactattatttaaacatttggttttaatggtaattatatataatgttaatgaactttcctataataatgcaaggttgaattgaaaattgttattatgatggggggattata

SEQ ID NO: 3 - DNA, 1896 bp, synthetic construct
  source 1..1896: mol_type = other DNA
atgaatgcaactatttgcaaattgaacaatgcagttatatccaagcccaatatagaactttcgcatagaagatgtaaaaagttcagtattgattcacaccaggtctttagtaaaaaaataatacacgatgctgaattacagaaaagatttgcagctaacagaaatttaatattgactgcagcaccttatatggagcagctaattaattttgtaaagggatttaatttttgtgtattacttacagatggagaaggatgcatattaaatgcgttaggtgatgaaaaaatactcgcagaagccttttctttgaaaatggtaccgggagcttttatgaatgaagaaaatattggaacaaatgctatgagcgtagttataaaaagtaagctgccagttcaaatttctggagatgatcattttattaatgcatatcataggtggacatgttctgcggcacctataaaagataataaaggaagacttataggtgtattgaatcttactggatatattgaatttgtacattctcatacacttggaatggttatagcagcgtcaaatgctatagaagaaatgcttaaagtagaagagtacaataaaattcaaaatatgaattacaagcatataaaaaatatatttaattccagtcctgttgctataataacttcagatataaatggtaaaataaaaatctgcaataaaaaggcacaagatatgtttggcataagaggtaataagttagagacaaacaatatggaagatattatagaaaattggaataatataaaaacacctatatatttaggagaaagtacttcaaaagaaacaattatggttgcactaagaaataagattcagtatcaagtaactactagttcaatatacaattgtgatgataataatattgagatagtctatgtttttgaaaaagctaaaaaggtaaataaaaaaaataatggccaggcttactatacctttggtaaaataatgggtcaggatgaaaactttacaaaagtaataaattatgcaaaaaagatttcagatagcaaatcaactatacttataatgggggaaagtggtacgggaaaagaagtgtttgcacaatccattcataattacagtagaagagtagatggaccatttgtagctttaaattgtggtgctatccctaaacaacttatagagtcagagctttttgggtatgaagaaggagcttttacaggtgccaaaaaaggtgggaaccttggaaaatttgaattagcagatggtggaactataatgctggatgaaattggagaaatgcctcttgatatgcagacaaagcttctaagagttgtacaggaaggcgtaataacgagaattggaagctcaaagtctatacctgtagatgtaagaattatagctgtcacaaacagaaatttaaaaaaagaagtggaatgtggaagatttagaaaagacttatattacagattaaatgttctgcctttattcttacctccacttagagaaagaaaaaaggatatacctcttcttattcaatattttgttaagaatatagcacaaaaattaaataaaaaagagccagttatatcagaagaatacttgaagaaaatgattaactataactggcctggaaatataagagaattagaaaatttggttgagctaattataaacactgaatccatacctacaggttactttacagaagaaaattgtgataatgaggtacttgtggatattagtgatgaatgtttaaagttagattatatggaaaaagaacatgtaactaaagtattgaagaaatttaaggggaatataacccattcagcagcagcactgggtataagaagaaatactttgtacagcaagattaaaaagtataatatagaagtgtaa

SEQ ID NO: 4 - DNA, 2883 bp, synthetic construct
  source 1..2883: mol_type = other DNA
agtttctaagctaaaaataaacttatatagagttcctaaacaaaaaatcaccttatattccggatattttttacaacattcagctcggttaaatattttgataagcctaagtaaaaaattctagaaaatctaagaacttttgaaaactcgtacctcaaacaattcaaagttctaagtttttacgaattttttactaagatttatttgcaaaatatttaaatgagcttcaccgttgtaaaaatatgcctacatataaggtgatttttaagttagaaagtgtggcataggtttatttttttagttataacacttgtatatttaattctacatagagtggagctgaagattgtaaaaatatctggagaataggttaatttctaacttagaagtctttcattaattccgtttatttttccactgggcgataagtacatatcctaaaaatataaggactaatcctataatcatagtaaaaaaagtttttataacattttcccaatttgatgcgttaaaagcacccatagtccagaatacaatagtaggaagttgctcataggggtcagctatgtattttaaaaaagaaagtcctgcaggaaaaagtgaagaaatgattattacagctaacactaatgtagttcaatatcctttaaattttgagcaacattaatgcaaaataaatgtaaataaatgatgatatttaaaatgcgttcgaaaaacgaactatagtcaaaaaacgaacacagcttttttaattgatttgatcaaaaagtgaacatacataatagaaaatataaaagaaactacctaatttgggcattttcaacaatggcatatatattgcttataagtaaataatgactctaaatttaaacatttaactattatttaaacatttggttttaatggtaattatatataatgttaatgaactttcctataataatgcaaggttgaattgaaaattgttattatgatggggggattataatgaatgcaactatttgcaaattgaacaatgcagttatatccaagcccaatatagaactttcgcatagaagatgtaaaaagttcagtattgattcacaccaggtctttagtaaaaaaataatacacgatgctgaattacagaaaagatttgcagctaacagaaatttaatattgactgcagcaccttatatggagcagctaattaattttgtaaagggatttaatttttgtgtattacttacagatggagaaggatgcatattaaatgcgttaggtgatgaaaaaatactcgcagaagccttttctttgaaaatggtaccgggagcttttatgaatgaagaaaatattggaacaaatgctatgagcgtagttataaaaagtaagctgccagttcaaatttctggagatgatcattttattaatgcatatcataggtggacatgttctgcggcacctataaaagataataaaggaagacttataggtgtattgaatcttactggatatattgaatttgtacattctcatacacttggaatggttatagcagcgtcaaatgctatagaagaaatgcttaaagtagaagagtacaataaaattcaaaatatgaattacaagcatataaaaaatatatttaattccagtcctgttgctataataacttcagatataaatggtaaaataaaaatctgcaataaaaaggcacaagatatgtttggcataagaggtaataagttagagacaaacaatatggaagatattatagaaaattggaataatataaaaacacctatatatttaggagaaagtacttcaaaagaaacaattatggttgcactaagaaataagattcagtatcaagtaactactagttcaatatacaattgtgatgataataatattgagatagtctatgtttttgaaaaagctaaaaaggtaaataaaaaaaataatggccaggcttactatacctttggtaaaataatgggtcaggatgaaaactttacaaaagtaataaattatgcaaaaaagatttcagatagcaaatcaactatacttataatgggggaaagtggtacgggaaaagaagtgtttgcacaatccattcataattacagtagaagagtagatggaccatttgtagctttaaattgtggtgctatccctaaacaacttatagagtcagagctttttgggtatgaagaaggagcttttacaggtgccaaaaaaggtgggaaccttggaaaatttgaattagcagatggtggaactataatgctggatgaaattggagaaatgcctcttgatatgcagacaaagcttctaagagttgtacaggaaggcgtaataacgagaattggaagctcaaagtctatacctgtagatgtaagaattatagctgtcacaaacagaaatttaaaaaaagaagtggaatgtggaagatttagaaaagacttatattacagattaaatgttctgcctttattcttacctccacttagagaaagaaaaaaggatatacctcttcttattcaatattttgttaagaatatagcacaaaaattaaataaaaaagagccagttatatcagaagaatacttgaagaaaatgattaactataactggcctggaaatataagagaattagaaaatttggttgagctaattataaacactgaatccatacctacaggttactttacagaagaaaattgtgataatgaggtacttgtggatattagtgatgaatgtttaaagttagattatatggaaaaagaacatgtaactaaagtattgaagaaatttaaggggaatataacccattcagcagcagcactgggtataagaagaaatactttgtacagcaagattaaaaagtataatatagaagtgtaa

SEQ ID NO: 5 - DNA, 3235 bp, synthetic construct
  source 1..3235: mol_type = other DNA
agtttctaagctaaaaataaacttatatagagttcctaaacaaaaaatcaccttatattccggatattttttacaacattcagctcggttaaatattttgataagcctaagtaaaaaattctagaaaatctaagaacttttgaaaactcgtacctcaaacaattcaaagttctaagtttttacgaattttttactaagatttatttgcaaaatatttaaatgagcttcaccgttgtaaaaatatgcctacatataaggtgatttttaagttagaaagtgtggcataggtttatttttttagttataacacttgtatatttaattctacatagagtggagctgaagattgtaaaaatatctggagaataggttaatttctaacttagaagtctttcattaattccgtttatttttccactgggcgataagtacatatcctaaaaatataaggactaatcctataatcatagtaaaaaaagtttttataacattttcccaatttgatgcgttaaaagcacccatagtccagaatacaatagtaggaagttgctcataggggtcagctatgtattttaaaaaagaaagtcctgcaggaaaaagtgaagaaatgattattacagctaacactaatgtagttcaatatcctttaaattttgagcaacattaatgcaaaataaatgtaaataaatgatgatatttaaaatgcgttcgaaaaacgaactatagtcaaaaaacgaacacagcttttttaattgatttgatcaaaaagtgaacatacataatagaaaatataaaagaaactacctaatttgggcattttcaacaatggcatatatattgcttataagtaaataatgactctaaatttaaacatttaactattatttaaacatttggttttaatggtaattatatataatgttaatgaactttcctataataatgcaaggttgaattgaaaattgttattatgatggggggattataatgaatgcaactatttgcaaattgaacaatgcagttatatccaagcccaatatagaactttcgcatagaagatgtaaaaagttcagtattgattcacaccaggtctttagtaaaaaaataatacacgatgctgaattacagaaaagatttgcagctaacagaaatttaatattgactgcagcaccttatatggagcagctaattaattttgtaaagggatttaatttttgtgtattacttacagatggagaaggatgcatattaaatgcgttaggtgatgaaaaaatactcgcagaagccttttctttgaaaatggtaccgggagcttttatgaatgaagaaaatattggaacaaatgctatgagcgtagttataaaaagtaagctgccagttcaaatttctggagatgatcattttattaatgcatatcataggtggacatgttctgcggcacctataaaagataataaaggaagacttataggtgtattgaatcttactggatatattgaatttgtacattctcatacacttggaatggttatagcagcgtcaaatgctatagaagaaatgcttaaagtagaagagtacaataaaattcaaaatatgaattacaagcatataaaaaatatatttaattccagtcctgttgctataataacttcagatataaatggtaaaataaaaatctgcaataaaaaggcacaagatatgtttggcataagaggtaataagttagagacaaacaatatggaagatattatagaaaattggaataatataaaaacacctatatatttaggagaaagtacttcaaaagaaacaattatggttgcactaagaaataagattcagtatcaagtaactactagttcaatatacaattgtgatgataataatattgagatagtctatgtttttgaaaaagctaaaaaggtaaataaaaaaaataatggccaggcttactatacctttggtaaaataatgggtcaggatgaaaactttacaaaagtaataaattatgcaaaaaagatttcagatagcaaatcaactatacttataatgggggaaagtggtacgggaaaagaagtgtttgcacaatccattcataattacagtagaagagtagatggaccatttgtagctttaaattgtggtgctatccctaaacaacttatagagtcagagctttttgggtatgaagaaggagcttttacaggtgccaaaaaaggtgggaaccttggaaaatttgaattagcagatggtggaactataatgctggatgaaattggagaaatgcctcttgatatgcagacaaagcttctaagagttgtacaggaaggcgtaataacgagaattggaagctcaaagtctatacctgtagatgtaagaattatagctgtcacaaacagaaatttaaaaaaagaagtggaatgtggaagatttagaaaagacttatattacagattaaatgttctgcctttattcttacctccacttagagaaagaaaaaaggatatacctcttcttattcaatattttgttaagaatatagcacaaaaattaaataaaaaagagccagttatatcagaagaatacttgaagaaaatgattaactataactggcctggaaatataagagaattagaaaatttggttgagctaattataaacactgaatccatacctacaggttactttacagaagaaaattgtgataatgaggtacttgtggatattagtgatgaatgtttaaagttagattatatggaaaaagaacatgtaactaaagtattgaagaaatttaaggggaatataacccattcagcagcagcactgggtataagaagaaatactttgtacagcaagattaaaaagtataatatagaagtgtaatatttttcattcgaaaaaaatagaggggttactggcaaatatattgttggtaaccctataattatgtttttattaatatggaatatatgggaaaaggaaaataatgaccaaaaaacgaacattaatcaaaaaatgaacaccattttttgattactttgatctaaaaatgaacacaacataataagcatcataaaaacactacctaatttggacactttcaacattggcatacatattgcttatataaatagtaacattaaatatttcgaccaacattaaaacgcatctacaatttgtaatatgaatgcgatttaaattgcaagttgtagataaaaaattgaggaggtatagt

SEQ ID NO: 6 - DNA, 3082 bp, synthetic construct
  source 1..3082: mol_type = other DNA
tatttttcattcgaaaaaaatagaggggttactggcaaatatattgttggtaaccctataattatgtttttattaatatggaatatatgggaaaaggaaaataatgaccaaaaaacgaacattaatcaaaaaatgaacaccattttttgattactttgatctaaaaatgaacacaacataataagcatcataaaaacactacctaatttggacactttcaacattggcatacatattgcttatataaatagtaacattaaatatttcgaccaacattaaaacgcatctacaatttgtaatatgaatgcgatttaaattgcaagttgtagataaaaaattgaggaggtatagtatgctaaaaaagtatttaattattaatggagttaaaaaaactattatagcagatccagagacatctttggcagatgttttaagaaagcaagcatttcttactggaacaaaagttggatgtagaaaaggagaatgtggtgcttgttccgtaattttgaatggtaaagtagtaagatcttgtataactaaaatgaaaagggttccagatgaatcagaaattataactatagaaggagttggcgatcaaaaacatcttcatccactgcagttagcatggatggttcatggtgcagcacagtgtggttattgcacacctggattcataatttcagctaaagtattacttgaacacaatagtaatccaacaagagaagatgttagagcctggttccaaaaacacagaaatgtttgtcgatgtacaggatataagccattagttgacgcagtaatggaagcagcaaaacttatgcgtggtgaaattacaaaagaagacatatggtatagacttccaaaaggaaaaagtcttttaggttcagaggctatacgtccttctgctcttgcaaaagtaacaggaaactgggattttggagcagatttaggattaaagatgccagaaaatacattacacattaagctagtacaagctacagtatctcatgcaaatatattatcgatagatacttcagaagcagaaaaaatggagggagtttatagagtactaacgtataaagatgtaccgggtacaaatagaataaatggattggctttccctacaaataagggtgatggacttgaaagacctatattaaatgataaaaagattttccaatttggtgatgcaatagctatggtacttgcagatacaccatgtcaagcagaagcagcagcaaaaaaagtaaaagttgaaatagaagaacttccagcatatatgagtgcaccagcagctatggctgaggatgctatagaaattcatccaggaacaccaaatatttattttgaatgtggtactgtaaaaggtgaagatacaaaacctttaatggataaattagaatatgttgttgaagatgattcatatgtacaaaggcagccgcatcttccacttgaaccagatgttggatgtgcatatattaatgaagatggaaagttaattgtacattcaaagagtgtagcacttcattgtcatgcagctatggtagctgacggtataggaatacctttaaaggatctttatatagttcaaaatcctacaggtggtactttcggatataaatttagtccaacaatggaaggacttctcggtgtagctgccttggtaactaaaagaccagtatatcttgagtttaatatgtatcaacaaataacttatactggaaaaagatcacctttctttatgcatcttaaattaggtgctgataaagcaggaaaattcactgcattagaaactgattggtcttgtgatcatggtccatattgtgaatttggtgaccttgtaactacaagaggttcacaatttatgggtgcaggatataatattccaaatataagaggagaaggaagaacagttgctacaaatcatgcttggggttcagcatttagaggatatggttcaccacaaagtttatttgcttcagaggttttagtagataaaatggctaagaaaatgggtgtggatcctcttgaattgagatataaaaatgtctatagagaaggagataaaacccctacaggttgtgacccagatgtaatatgcttgccaactatgattgaaaagttacgtccattatataaggatgctaaggaaagagttaaaaataaaaatgttaattctggagataagaaatatggagtaggaacttccttattaatttatagttgtggccttgatggtgcagatagttcaaatgcatgggcagaattgactgcagaaggggttactgtaggaaactcttggcaagatcacggtcaaggagctgacatgggaactttaactatagctcatgaaactttaagaccaataggattagagccagaagatattaagttagttatgaatgatatggaatttactccagacagtggtccttcaggtggaagccgttctaacgttttaacagggaatgctactagagttgcttgtgaaaacttattagcagctatgaaaaaagatgacggtacttacatgacttatgatgaaatggtagctgcaaaaaaacctttaaaatatgatggaaaatggacagcaccttgtacagctcctgatccagcaactggtcaaggagatccattcccaacttatatgtatggtgtccttatgtcagaagtagaagtagatactactacaggaaagactaaagtagaaaaacttactctaatagctgatgtaggaactatagtaaataaactagtagttgatggtcaaatgtatggtggactagcccaaggtataggacttgctttaagtgaagattttgaagatttaaagaaacatactacacttacaggttgtggaataccgcaaattagggatgtacctgatgatataaaaataatatatcaagaaacaccacgaccaaatggcccttatggtgcttcaggtgcaggtgaaatgccactttcagcacctcatgcttctatagttaatgcaatttatgatgcttgtggagtacagataacaaaattacctgcacgtccagaaaaagttttagcagcactaaaagaattaaaaaaataa

SEQ ID NO: 7 - DNA, 5965 bp, synthetic construct
  source 1..5965: mol_type = other DNA
agtttctaagctaaaaataaacttatatagagttcctaaacaaaaaatcaccttatattccggatattttttacaacattcagctcggttaaatattttgataagcctaagtaaaaaattctagaaaatctaagaacttttgaaaactcgtacctcaaacaattcaaagttctaagtttttacgaattttttactaagatttatttgcaaaatatttaaatgagcttcaccgttgtaaaaatatgcctacatataaggtgatttttaagttagaaagtgtggcataggtttatttttttagttataacacttgtatatttaattctacatagagtggagctgaagattgtaaaaatatctggagaataggttaatttctaacttagaagtctttcattaattccgtttatttttccactgggcgataagtacatatcctaaaaatataaggactaatcctataatcatagtaaaaaaagtttttataacattttcccaatttgatgcgttaaaagcacccatagtccagaatacaatagtaggaagttgctcataggggtcagctatgtattttaaaaaagaaagtcctgcaggaaaaagtgaagaaatgattattacagctaacactaatgtagttcaatatcctttaaattttgagcaacattaatgcaaaataaatgtaaataaatgatgatatttaaaatgcgttcgaaaaacgaactatagtcaaaaaacgaacacagcttttttaattgatttgatcaaaaagtgaacatacataatagaaaatataaaagaaactacctaatttgggcattttcaacaatggcatatatattgcttataagtaaataatgactctaaatttaaacatttaactattatttaaacatttggttttaatggtaattatatataatgttaatgaactttcctataataatgcaaggttgaattgaaaattgttattatgatggggggattataatgaatgcaactatttgcaaattgaacaatgcagttatatccaagcccaatatagaactttcgcatagaagatgtaaaaagttcagtattgattcacaccaggtctttagtaaaaaaataatacacgatgctgaattacagaaaagatttgcagctaacagaaatttaatattgactgcagcaccttatatggagcagctaattaattttgtaaagggatttaatttttgtgtattacttacagatggagaaggatgcatattaaatgcgttaggtgatgaaaaaatactcgcagaagccttttctttgaaaatggtaccgggagcttttatgaatgaagaaaatattggaacaaatgctatgagcgtagttataaaaagtaagctgccagttcaaatttctggagatgatcattttattaatgcatatcataggtggacatgttctgcggcacctataaaagataataaaggaagacttataggtgtattgaatcttactggatatattgaatttgtacattctcatacacttggaatggttatagcagcgtcaaatgctatagaagaaatgcttaaagtagaagagtacaataaaattcaaaatatgaattacaagcatataaaaaatatatttaattccagtcctgttgctataataacttcagatataaatggtaaaataaaaatctgcaataaaaaggcacaagatatgtttggcataagaggtaataagttagagacaaacaatatggaagatattatagaaaattggaataatataaaaacacctatatatttaggagaaagtacttcaaaagaaacaattatggttgcactaagaaataagattcagtatcaagtaactactagttcaatatacaattgtgatgataataatattgagatagtctatgtttttgaaaaagctaaaaaggtaaataaaaaaaataatggccaggcttactatacctttggtaaaataatgggtcaggatgaaaactttacaaaagtaataaattatgcaaaaaagatttcagatagcaaatcaactatacttataatgggggaaagtggtacgggaaaagaagtgtttgcacaatccattcataattacagtagaagagtagatggaccatttgtagctttaaattgtggtgctatccctaaacaacttatagagtcagagctttttgggtatgaagaaggagcttttacaggtgccaaaaaaggtgggaaccttggaaaatttgaattagcagatggtggaactataatgctggatgaaattggagaaatgcctcttgatatgcagacaaagcttctaagagttgtacaggaaggcgtaataacgagaattggaagctcaaagtctatacctgtagatgtaagaattatagctgtcacaaacagaaatttaaaaaaagaagtggaatgtggaagatttagaaaagacttatattacagattaaatgttctgcctttattcttacctccacttagagaaagaaaaaaggatatacctcttcttattcaatattttgttaagaatatagcacaaaaattaaataaaaaagagccagttatatcagaagaatacttgaagaaaatgattaactataactggcctggaaatataagagaattagaaaatttggttgagctaattataaacactgaatccatacctacaggttactttacagaagaaaattgtgataatgaggtacttgtggatattagtgatgaatgtttaaagttagattatatggaaaaagaacatgtaactaaagtattgaagaaatttaaggggaatataacccattcagcagcagcactgggtataagaagaaatactttgtacagcaagattaaaaagtataatatagaagtgtaatatttttcattcgaaaaaaatagaggggttactggcaaatatattgttggtaaccctataattatgtttttattaatatggaatatatgggaaaaggaaaataatgaccaaaaaacgaacattaatcaaaaaatgaacaccattttttgattactttgatctaaaaatgaacacaacataataagcatcataaaaacactacctaatttggacactttcaacattggcatacatattgcttatataaatagtaacattaaatatttcgaccaacattaaaacgcatctacaatttgtaatatgaatgcgatttaaattgcaagttgtagataaaaaattgaggaggtatagtatgctaaaaaagtatttaattattaatggagttaaaaaaactattatagcagatccagagacatctttggcagatgttttaagaaagcaagcatttcttactggaacaaaagttggatgtagaaaaggagaatgtggtgcttgttccgtaattttgaatggtaaagtagtaagatcttgtataactaaaatgaaaagggttccagatgaatcagaaattataactatagaaggagttggcgatcaaaaacatcttcatccactgcagttagcatggatggttcatggtgcagcacagtgtggttattgcacacctggattcataatttcagctaaagtattacttgaacacaatagtaatccaacaagagaagatgttagagcctggttccaaaaacacagaaatgtttgtcgatgtacaggatataagccattagttgacgcagtaatggaagcagcaaaacttatgcgtggtgaaattacaaaagaagacatatggtatagacttccaaaaggaaaaagtcttttaggttcagaggctatacgtccttctgctcttgcaaaagtaacaggaaactgggattttggagcagatttaggattaaagatgccagaaaatacattacacattaagctagtacaagctacagtatctcatgcaaatatattatcgatagatacttcagaagcagaaaaaatggagggagtttatagagtactaacgtataaagatgtaccgggtacaaatagaataaatggattggctttccctacaaataagggtgatggacttgaaagacctatattaaatgataaaaagattttccaatttggtgatgcaatagctatggtacttgcagatacaccatgtcaagcagaagcagcagcaaaaaaagtaaaagttgaaatagaagaacttccagcatatatgagtgcaccagcagctatggctgaggatgctatagaaattcatccaggaacaccaaatatttattttgaatgtggtactgtaaaaggtgaagatacaaaacctttaatggataaattagaatatgttgttgaagatgattcatatgtacaaaggcagccgcatcttccacttgaaccagatgttggatgtgcatatattaatgaagatggaaagttaattgtacattcaaagagtgtagcacttcattgtcatgcagctatggtagctgacggtataggaatacctttaaaggatctttatatagttcaaaatcctacaggtggtactttcggatataaatttagtccaacaatggaaggacttctcggtgtagctgccttggtaactaaaagaccagtatatcttgagtttaatatgtatcaacaaataacttatactggaaaaagatcacctttctttatgcatcttaaattaggtgctgataaagcaggaaaattcactgcattagaaactgattggtcttgtgatcatggtccatattgtgaatttggtgaccttgtaactacaagaggttcacaatttatgggtgcaggatataatattccaaatataagaggagaaggaagaacagttgctacaaatcatgcttggggttcagcatttagaggatatggttcaccacaaagtttatttgcttcagaggttttagtagataaaatggctaagaaaatgggtgtggatcctcttgaattgagatataaaaatgtctatagagaaggagataaaacccctacaggttgtgacccagatgtaatatgcttgccaactatgattgaaaagttacgtccattatataaggatgctaaggaaagagttaaaaataaaaatgttaattctggagataagaaatatggagtaggaacttccttattaatttatagttgtggccttgatggtgcagatagttcaaatgcatgggcagaattgactgcagaaggggttactgtaggaaactcttggcaagatcacggtcaaggagctgacatgggaactttaactatagctcatgaaactttaagaccaataggattagagccagaagatattaagttagttatgaatgatatggaatttactccagacagtggtccttcaggtggaagccgttctaacgttttaacagggaatgctactagagttgcttgtgaaaacttattagcagctatgaaaaaagatgacggtacttacatgacttatgatgaaatggtagctgcaaaaaaacctttaaaatatgatggaaaatggacagcaccttgtacagctcctgatccagcaactggtcaaggagatccattcccaacttatatgtatggtgtccttatgtcagaagtagaagtagatactactacaggaaagactaaagtagaaaaacttactctaatagctgatgtaggaactatagtaaataaactagtagttgatggtcaaatgtatggtggactagcccaaggtataggacttgctttaagtgaagattttgaagatttaaagaaacatactacacttacaggttgtggaataccgcaaattagggatgtacctgatgatataaaaataatatatcaagaaacaccacgaccaaatggcccttatggtgcttcaggtgcaggtgaaatgccactttcagcacctcatgcttctatagttaatgcaatttatgatgcttgtggagtacagataacaaaattacctgcacgtccagaaaaagttttagcagcactaaaagaattaaaaaaataa

(19) United States
(12) Patent Application Publication
(10) Pub. No.: US 2025/0084445 A1
(43) Pub. Date: Mar. 13, 2025

(54) ORGANISMS AND METHODS OF USE THEREOF

(71) Applicant: SYNATA BIO, INC., Warrenville, IL (US)

(72) Inventors: David Harvey KEATING, Warrenville, IL (US); Matthew Theodore KISSINGER, Warrenville, IL (US)

(73) Assignee: SYNATA BIO, INC., Warrenville, IL (US)

(21) Appl. No.: 18/728,382

(22) PCT Filed: Jan. 11, 2023
(86) PCT No.: PCT/US2023/060496

§ 371 Date: Jul. 11, 2024

Related U.S. Application Data
(60) Provisional application No. 63/298,444, filed on Jan. 11, 2022.

(51) Int. Cl. C12P 7/40; C12N 9/02; C12N 15/74

(52) U.S. Cl. CPC C12P 7/40; C12N 9/0008; C12N 15/74; C12Y 102/03001; C12N 2800/101

(57) ABSTRACT

Disclosed are organisms comprising a highly expressed form of EC 1.2.3.1, and methods of their use including methods for efficient fermentation broth recycle, methods for improving bottoms recycle, methods for converting CO, CO₂, and optionally H₂ to ethanol and other oxygenated products, methods for preparing animal feed, and methods for preparing fertilizer.

### CROSS REFERENCE TO RELATED APPLICATIONS

This patent application claims the benefit of co-pending U.S. Provisional Patent Application No. 63/298,444, filed Jan. 11, 2022, which is incorporated by reference herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 25,241 Byte XML file named “766307.xml,” dated Jan. 9, 2023.

### BACKGROUND OF THE INVENTION

Enzymes belonging to EC 1.2.3.1 are aldehyde dehydrogenases, and these enzymes are useful for interconverting aldehydes and acids. Most of the enzymes in this class studied to date carry out the addition of H₂O and O₂ to carboxylates and H₂O₂. However, enzymes of this class are also found in obligate anaerobes (Bradshaw et al., J. Biol Chem., 235: 3620-3629 (1980)). It has been suggested that, under anaerobic conditions, the primary biological function of these enzymes is the reduction of carboxylates. When the conversion of carboxylates is desired, one option is to have organisms express higher than naturally occurring levels of enzymes belonging to EC 1.2.3.1 to increase the rate of these reactions.

It is also desirable to use organisms to convert gases containing carbon monoxide (CO) and carbon dioxide (CO₂), and optionally hydrogen (H₂), such as industrial waste gas or syngas, into a variety of products, such as fuels and chemicals using fermentation. The low ethanol tolerance of organisms that can consume CO, CO₂, and H₂ requires the use of continuous fermentation techniques, whereby broth containing ethanol, acetate, and organisms (and components thereof) are removed from the fermentor and new growth medium is added. The ethanol and organisms (and components thereof) are then recovered from the removed broth. The ethanol-depleted removed broth then needs to be subjected to wastewater treatment prior to release, which adds significantly to capital and process costs. For example, a commercial-scale bioreactor may contain in excess of 1 million liters of aqueous broth, and creating a wastewater treatment system to handle all of the ethanol-depleted removed broth would require significant capital costs.

Accordingly, there remains a need for methods that reduce the need for expensive wastewater treatment and high water use associated with continuous fermentation. One approach to reducing this burden on wastewater treatment is to recycle the ethanol-depleted removed broth back to the fermentor (referred to as bottoms recycle). However, the percentage of ethanol-depleted removed broth that can be re-introduced to the fermentor is limited by the tolerance of the organism for this recycled broth. Therefore, improved organisms that permit a greater portion of removed broth to be recycled are advantageous.

### BRIEF SUMMARY OF THE INVENTION

An aspect of the invention provides organisms comprising a highly expressed form of EC 1.2.3.1.

Another aspect of the invention provides methods for efficient fermentation broth recycle, the methods comprising providing to a bioreactor a gaseous substrate comprising CO, CO₂, and optionally H₂, at least one organism of an aspect of the invention, and a liquid nutrient medium, and providing conditions within the bioreactor for the at least one organism to convert CO, CO₂, and optionally H₂ to at least one oxygenated product, wherein the conditions within the bioreactor create a bioreactor broth.

A further aspect of the invention provides methods for improving bottoms recycle, the methods comprising providing to a bioreactor a gaseous substrate comprising CO, CO₂, and optionally H₂, at least one organism of an aspect of the invention, and a liquid nutrient medium, and providing conditions within the bioreactor for the at least one organism to convert CO, CO₂, and optionally H₂ to at least one oxygenated product, wherein the conditions within the bioreactor create a bioreactor broth.

An additional aspect of the invention provides methods for converting CO, CO₂, and optionally H₂ to ethanol, the methods comprising providing to a bioreactor a gaseous substrate comprising CO, CO₂, and optionally H₂, at least one organism of an aspect of the invention, and a liquid nutrient medium, and providing conditions within the bioreactor for the at least one organism to convert CO, CO₂, and optionally H₂ to at least one oxygenated product, wherein the conditions within the bioreactor create a bioreactor broth.

Additional aspects include methods for preparing animal feed and for preparing fertilizer.

### BRIEF DESCRIPTION OF THE SEVERAL VIEWS OF THE DRAWING(S)

FIG. 1 is a bar graph showing the level of expression of non-tungsten aldehyde oxidoreductase (EC 1.2.3.1 class; Non-W AOR), alcohol dehydrogenase (ADH), and the two copies of the tungsten containing AOR (EC 1.2.7.5; W-AOR I and W-AOR II) in organism SB1.

FIG. 2 is a bar graph showing the level of expression of non-tungsten aldehyde oxidoreductase (EC 1.2.3.1 class; Non-W AOR), alcohol dehydrogenase (ADH), and the two copies of the tungsten containing AOR (EC 1.2.7.5; W-AOR I and W-AOR II) in Clostridium autoethanogenum.

FIG. 3 is an image showing plots of the mean read density of a gene cluster surrounding the two copies of the aldehyde oxidoreductase EC 1.2.3.1 in organism SB1. Higher lines on the plots are proportional to higher levels of gene expression.

FIG. 4 is an image showing plots of the mean read density of a gene cluster around the single copy of the aldehyde oxidoreductase EC 1.2.3.1 in Clostridium autoethanogenum. Higher lines on the plots are proportional to higher levels of gene expression.

FIG. 5 is a schematic of the Wood-Ljungdahl pathway. The role of the aldehyde oxidoreductase (AOR) and ADH in converting acetate and acetaldehyde, respectively, are shown.

FIG. 6A is a schematic of plasmid pKO32.

FIG. 6B is an image of petri dish showing multiple small colonies growing following conjugation and antibiotic selection.

FIG. 6C is a set of gels showing an aspect of the present invention used to confirm the identification of organism (top) and presence of plasmid pKO32 in the organism (bottom).

FIG. 6D is a gel showing that the organisms of FIG. 6C had pKO32 plasmids that were not integrated into the highly expressed non-tungsten AOR gene.

### DETAILED DESCRIPTION OF THE INVENTION

An aspect of the invention provides organisms comprising a highly expressed form of EC 1.2.3.1. As used herein, “a highly expressed form of EC 1.2.3.1” means that the enzyme belonging to EC 1.2.3.1 is expressed at a level equal to or exceeding the expression level of enzyme EC 1.2.7.5 in the organism.

In an aspect, the the organisms are non-naturally occurring organisms that contain a gene encoding an enzyme belonging to EC 1.2.3.1. In an aspect, the gene is an “exogenous gene,” meaning a gene which originates outside of the organism to which the gene is introduced. Exogenous genes may be derived from any appropriate source, including, but not limited to, the organisms to which they are to be introduced (for example, a parental organism from which the recombinant organism is derived), strains or species of organisms which differ from the organism to which they are to be introduced, or they may be artificially or recombinantly created. In one aspect, the exogenous gene is introduced to increase expression of or over-express a particular gene (for example, by increasing the copy number of the gene), or introducing a strong or constitutive promoter to increase expression). In another aspect, the exogenous gene represents a gene not naturally present within the organism to which the gene is to be introduced and allows for the expression of a product not naturally present within the organism or increased expression of a gene native to the organism (for example, in the case of introduction of a regulatory element such as a promoter). The exogenous gene may be adapted to integrate into the genome of the organism to which the exogenous gene is to be introduced.

In a further aspect, the gene is an endogenous gene.

“Non-naturally occurring” as used herein refers to an organism that has been modified by the hand of man and has at least one genetic modification not found in a naturally occurring strain of the referenced species, i.e., not found in the wild-type strain of the referenced species.

In an aspect, the enzyme belonging to EC 1.2.3.1 is an aldehyde oxidoreductase (AOR).

In an aspect, the organism is a microorganism. In an aspect, the organism is an archea, bacterium, or yeast. In a further aspect, the organism is a bacterium. In yet another aspect, the organism is an acetogenic carboxydotrophic bacterium. In an aspect, the organism is a Clostridium bacterium.

In an aspect, the organism has increased activity of an enzyme belonging to EC 1.2.3.1 as compared to an organism that is the same except for not having a gene encoding an enzyme belonging to EC 1.2.3.1.

In another aspect, the organism has increased activity of an enzyme belonging to EC 1.2.3.1 due to an increased number of copies of a gene encoding an enzyme belonging to EC 1.2.3.1 and/or increased promoter activity of a gene encoding an enzyme belonging to EC 1.2.3.1.

In an aspect, the at least one organism comprises a sequence with at least about 95%, about 96%, about 97%, about 98%, about 99%, or about 100% identity to any one of SEQ ID NOs: 1-7.

In an aspect, the at least one organism comprises a sequence with at least about 95%, about 96%, about 97%, about 98%, about 99%, or about 100% identity to SEQ ID NO: 1.

In an aspect, the at least one organism comprises a sequence with at least about 95%, about 96%, about 97%, about 98%, about 99%, or about 100% identity to SEQ ID NO: 2.

In an aspect, the at least one organism comprises a sequence with at least about 95%, about 96%, about 97%, about 98%, about 99%, or about 100% identity to SEQ ID NO: 3.

In an aspect, the at least one organism comprises a sequence with at least about 95%, about 96%, about 97%, about 98%, about 99%, or about 100% identity to SEQ ID NO: 4.

In an aspect, the at least one organism comprises a sequence with at least about 95%, about 96%, about 97%, about 98%, about 99%, or about 100% identity to SEQ ID NO: 5.

In an aspect, the at least one organism comprises a sequence with at least about 95%, about 96%, about 97%, about 98%, about 99%, or about 100% identity to SEQ ID NO: 6.

In an aspect, the at least one organism comprises a sequence with at least about 95%, about 96%, about 97%, about 98%, about 99%, or about 100% identity to SEQ ID NO: 7.

As noted above, one approach to reducing wastewater treatment costs and high water usage associated with continuous fermentation is to recycle the removed broth back to the reactor. Acetic acid (“acetic acid” and “acetate” are used interchangeably herein) is also produced during fermentation of CO, CO₂, and H₂ and, unlike ethanol, is not completely removed from the fermentor broth by methods such as distillation. Therefore, in the absence of conversion by the organism, the acetate in the media will increase during fermentor broth recycling, which will lead to toxicity and reduced fermentation performance. Although the fermentation broth could be discarded in the event that the concentration of acetate becomes excessive, nutrients for the fermentation would also be lost. In addition, the acetate represents lost revenue, and an increased burden on wastewater treatment.

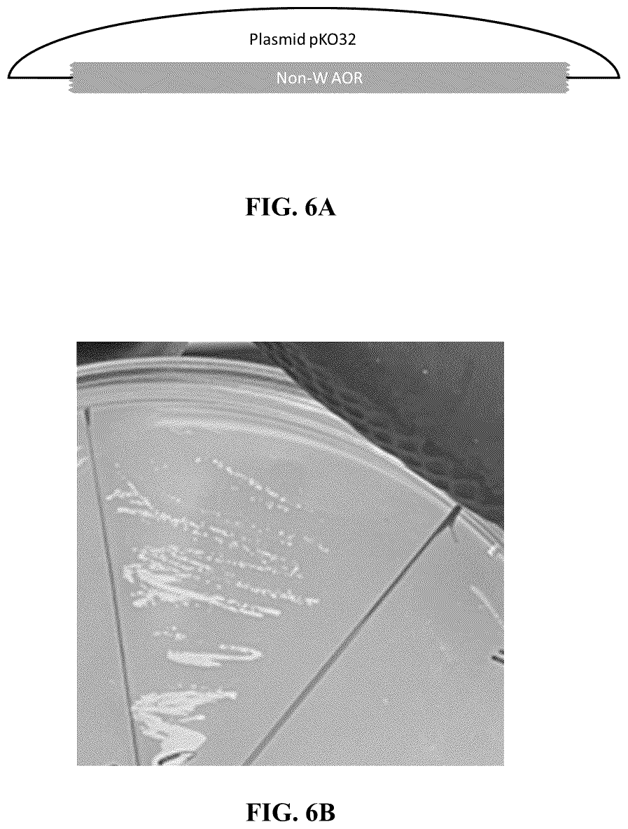

FIG. 5 illustrates the Wood-Ljungdahl pathway during production of ethanol from syngas. The Wood-Ljungdahl pathway converts CO, CO₂, and optionally H₂ to acetyl-CoA. The acetyl-CoA is then converted to acetate. The resulting acetate can then be converted to ethanol via the activities of aldehyde oxidoreductases (AOR) and alcohol dehydrogenase (ADH). The AOR activity converting acetate to acetaldehyde can be carried out by the enzymes of the EC 1.2.7.5 or EC 1.2.3.1 class. In the case of enzymes of the tungsten AOR class (EC 1.2.7.5), the cofactor used is reduced ferredoxin. In the case of non-tungsten AOR class (EC 1.2.3.1) found in anaerobes, the cofactor has not been determined. Acetate (in the form of acetic acid) that is returned to the fermenter during broth recycle can enter the cell and be converted to ethanol by these enzymes.

A key limitation to tolerance is the presence of carboxylates in the recycled broth. Carboxylates such as acetic acid are not completely eliminated from the broth during removal of ethanol by distillation. These carboxylates remain in the broth and are returned to the reactor during broth recycle. Organisms with a greater capacity to convert the carboxylates in recycled broth to alcohols will be able to tolerate a higher percentage of recycled broth.

Conversion of carboxylates to alcohols is carried out in Clostridia in two steps in syngas-utilizing Clostridia. The first step involves the reduction of the carboxylate to the corresponding aldehyde, which is then reduced to the alcohol via an alcohol dehydrogenase (Mock et al., J. Bacteriol., 197: 2965-2980 (2015)). The best studied enzyme that carries out this step is the tungsten-containing aldehyde oxidoreductases (EC 1.2.7.5: referred to herein as tungsten-containing AOR). These enzymes use ferredoxin as a cofactor. Typically, multiple copies of this class of enzymes are found in syngas-utilizing Clostridia, with differing levels of expression. Unexpectedly, it was found that Clostridia with high expression levels of the aldehyde oxidoreductases of the 1.2.3.1 class (referred to herein as non-W aldehyde oxidoreductases) have a greater ability to convert carboxylates to alcohols, and a greater ability to tolerate ethanol-depleted bottoms recycle. See U.S. Provisional Patent Application 63/136,025, incorporated herein by reference.

It has been discovered that an increase in efficiency of fermentation broth recycle can be achieved by using organisms comprising a highly expressed form of EC 1.2.3.1. The organism may contain a gene (e.g., exogenous gene) encoding an enzyme belonging to EC 1.2.3.1.

An aspect of the invention provides methods for efficient fermentation broth recycle, the methods comprising (a) providing to a bioreactor (1) a gaseous substrate comprising CO, CO₂, and optionally H₂, (2) at least one organism of an aspect of the invention, and (3) a liquid nutrient medium, and (b) providing conditions within the bioreactor for the at least one organism to convert CO, CO₂, and optionally H₂ to at least one oxygenated product, wherein the conditions within the bioreactor create a bioreactor broth.

In an aspect, the oxygenated product is acetic acid, butyrate, butanol, propionate, and propanol. In an aspect, the oxygenated product is ethanol.

In an aspect, the gaseous substrate comprising CO, CO₂, and optionally H₂ is a synthesis gas (syngas), such as syngas obtained by gasification of coal or refinery residues, gasification of biomass or lignocellulosic material, or reforming of natural gas. In another aspect, the syngas may be obtained from the gasification of municipal solid waste or industrial solid waste.

The gaseous substrate may comprise at least CO, such as about 1, about 2, about 5, about 10, about 20, about 30, about 40, about 50, about 60, about 70, about 80, about 90, or about 100 mol % CO. The gaseous substrate may comprise a range of CO, such as about 20 to about 80, about 30 to about 70, or about 40 to about 60 mol % CO. Preferably, the substrate comprises about 40 to about 70 mol % CO (e.g., steel mill or blast furnace gas), about 20 to about 30 mol % CO (e.g., basic oxygen furnace gas), or about 15 to about 45 mol % CO (e.g., syngas). In some aspects, the gaseous substrate may comprise a relatively low amount of CO, such as about 1 to about 10 or about 1 to about 20 mol % CO. In some aspects, the gaseous substrate comprises no or substantially no (<about 1 mol %) CO.

The gaseous substrate may optionally comprise H₂. For example, the gaseous substrate may comprise about 1, about 2, about 5, about 10, about 15, about 20, or about 30 mol % H₂. In some aspects, the gaseous substrate may comprise a relatively high amount of H₂, such as about 60, about 70, about 80, or about 90 mol % H₂. In another aspect, the gaseous substrate comprises no or substantially no (<about 1 mol %) H₂ (e.g., when derived from steel mill gas). The H₂ may be derived from or produced by any suitable process, including the formation of H₂ using electrodes.

The gaseous substrate may comprise CO₂. For example, the gaseous substrate may comprise from about 1 to about 80, from about 1 to about 70, from about 1 to about 60, from about 1 to about 50, from about 1 to about 40, from about 1 to about 30, from about 1 to about 25, from about 1 to about 20, from about 1 to about 15, from about 1 to about 10, from about 1 to about 5, from about 1 to about 4, from about 1 to about 3, or from about 2 to about 3 mol % CO₂. In some aspects, the gaseous substrate may comprise less than about 20, about 15, about 10, or about 5 mol % CO₂. In another aspect, the gaseous substrate comprises no or substantially no (<about 1 mol %) CO₂.

In an aspect, the gene encoding an enzyme belonging to EC 1.2.3.1 in the at least one organism is expressed at least about 1.5, at least about 1.75, at least about 2, at least about 2.25, at least about 2.5, at least about 2.75, at least about 3, at least about 3.25, at least about 3.5, at least about 3.75, at least about 4, at least about 4.25, or at least about 5 times more as compared to an organism that is the same except for not having a gene encoding an enzyme belonging to EC 1.2.3.1, when the at least one organism and the organism that does not have a gene encoding an enzyme belonging to EC 1.2.3.1 are cultured under similar culturing conditions.

In an aspect, the exogenous gene encoding an enzyme belonging to EC 1.2.3.1 in the at least one organism is expressed at least about 1.5, at least about 1.75, at least about 2, at least about 2.25, at least about 2.5, at least about 2.75, at least about 3, at least about 3.25, at least about 3.5, at least about 3.75, at least about 4, at least about 4.25, or at least about 5 times more as compared to an organism that is the same except for not having an exogenous gene encoding an enzyme belonging to EC 1.2.3.1, when the at least one organism and the organism that does not have an exogenous gene encoding an enzyme belonging to EC 1.2.3.1 are cultured under similar culturing conditions.

In an aspect, at least about 5%, at least about 6%, at least about 7%, at least about 8%, at least about 9%, at least about 10%, at least about 15%, at least about 20%, or at least about 25% more of the at least one oxygenated product is produced by the organism as compared to the amount of the at least one oxygenated product produced by an organism that is the same except for not having a gene encoding an enzyme belonging to EC 1.2.3.1 when the at least one organism and the organism that does not have a gene encoding an enzyme belonging to EC 1.2.3.1 are cultured under similar culturing conditions.

In an aspect, at least about 5%, at least about 6%, at least about 7%, at least about 8%, at least about 9%, at least about 10%, at least about 15%, at least about 20%, or at least about 25% more of the at least one oxygenated product is produced by the organism as compared to the amount of the at least one oxygenated product produced by an organism that is the same except for not having an exogenous gene encoding an enzyme belonging to EC 1.2.3.1 when the at least one organism and the organism that does not have an exogenous gene encoding an enzyme belonging to EC 1.2.3.1 are cultured under similar culturing conditions.

In an aspect, the organism is cultured in the bioreactor to produce a culture. In an aspect, the culture continuously produces at least one oxygenated product for more than about 24 hours, about 36 hours, about 48 hours, about 72 hours, about 96 hours, about 120 hours, about 144 hours, about 168 hours, about 192 hours, about 216 hours, about 250 hours, about 300 hours, about 400 hours, about 500 hours, about 600 hours, about 700 hours, about 800 hours, about 900 hours, about 1,000, 1,100 hours, about 1,200 hours, about 1,300 hours, about 1,400 hours, about 1,500 hours, about 1,600 hours, about 1,700 hours, about 1,800 hours, about 1,900 hours, about 2,000 hours, about 2,500 hours, or about 3,000 hours.

In an aspect, the inventive methods further comprise removing the bioreactor broth from the bioreactor to produce a removed broth.

In an aspect, the inventive methods further comprise removing the at least one oxygenated product from the removed broth to produce an oxygenated product-depleted removed broth. The oxygenated products may be separated or purified from the fermentation broth using any method or combination of methods known in the art, including, for example, fractional distillation, evaporation, pervaporation, gas stripping, phase separation, and extractive fermentation, including for example, liquid-liquid extraction. In certain aspects, target products are recovered from the fermentation broth by continuously removing a portion of the broth from the bioreactor, recovering one or more oxygenated products from the fermentation broth, and separating cells (and components thereof) from the broth (e.g., filtration). Alcohols may be recovered, for example, by distillation.

In an aspect, the inventive methods further comprise removing cells (and components thereof) of the culture from the removed broth and/or the oxygenated product-depleted removed broth. Removal of the cells (and components thereof) can be carried out in any suitable means, for example, by cyclones, filtration, or centrifugation. In an aspect, the majority (i.e., greater than 50%) of the cells (and components thereof) of the culture are removed. In an aspect, the removed cells (and components thereof) of the culture are removed from the removed broth before one or more oxygenated products are recovered from the fermentation broth. In an aspect, the removed cells (and components thereof) of the culture are removed from the removed broth after one or more oxygenated products are recovered from the fermentation broth.

In an aspect, the inventive methods further comprise providing the oxygenated product-depleted removed broth to the bioreactor. Additional nutrients (e.g. B vitamins and metals) may be added to the broth to replenish the broth before the broth is returned to the bioreactor.

In an aspect, the inventive methods further comprise providing the oxygenated product-depleted removed broth and the removed broth to the bioreactor. Additional nutrients (e.g. B vitamins and metals) may be added to the broths to replenish the broths before the broths are returned to the bioreactor.

In an aspect, at least about 1 gram, about 2 grams, about 3 grams, about 4 grams, about 5 grams, about 6 grams, about 7 grams, about 8 grams, about 9 grams, about 10 grams, about 11 grams, about 12 grams, about 13 grams, about 14 grams, about 15 grams, or about 20 grams, or about 25 grams, or about 30 grams, or about 35 grams, or from about 16 to about 20 grams of the at least one oxygenated product are produced per liter of removed broth.

In an aspect, more than about 30%, about 35%, about 40%, about 45%, about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor. In this regard, from about 1% to about 100% of the oxygenated product-depleted removed broth is provided back to the bioreactor, for example, from about 1% to about 30%, from about 1% to about 35%, from about 1% to about 40%, from about 1% to about 45%, from about 1% to about 50%, from about 1% to about 55%, from about 1% to about 60%, from about 1% to about 65%, from about 1% to about 70%, from about 1% to about 75%, or from about 1% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the removed broth is provided back to the bioreactor. In an alternative aspect, from about 50% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor.

An aspect of the invention further provides methods for improving bottoms recycle, the methods comprising (a) providing to a bioreactor (1) a gaseous substrate comprising CO, CO₂, and optionally H₂, (2) at least one organism of the invention, and (3) a liquid nutrient medium, and (b) providing conditions within the bioreactor for the at least one organism to convert CO, CO₂, and optionally H₂ to at least one oxygenated product, wherein the conditions within the bioreactor create a bioreactor broth.

An aspect of the invention further provides methods for converting CO, CO₂ and optionally H₂ to ethanol, the methods comprising (a) providing to a bioreactor (1) a gaseous substrate comprising CO, CO₂, and optionally H₂, (2) at least one organism of the invention, and (3) a liquid nutrient medium, and (b) providing conditions within the bioreactor for the at least one organism to convert CO, CO₂, and optionally H₂ to at least one oxygenated product, wherein the conditions within the bioreactor create a bioreactor broth.

As used herein, the term “promoter” refers to an untranscribed sequence located upstream (i.e., 5′) to the translation start codon of a gene (generally within about 1 to 1000 bp, preferably 1-500 bp, especially 1-100 bp) and which controls or influences the start of transcription of the gene.

“Increasing the efficiency,” “increased efficiency,” and the like include, but are not limited to, increasing the amount of oxygenated product-depleted removed broth that can be provided to the bioreactor without negatively impacting growth of the at least one acetogenic carboxydotrophic bacterium.

Typically the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In certain aspects, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

As used herein, a “bioreactor” refers to a bioreactor assembly. A bioreactor assembly is a group of one or more vessels suitable to contain aqueous broth and organisms for the bioconversion. The bioreactor assembly may contain associated equipment such as injectors, recycle loops, and agitators. As used herein, “providing the oxygenated product-depleted removed broth to the bioreactor” means that the oxygenated product-depleted removed broth can be added to any portion of a bioreactor assembly. For example, the oxygenated product-depleted removed broth may be added to a recycle loop, to the vessels that contain aqueous broth, or to any convenient location within the bioreactor assembly. The oxygenated product-depleted removed broth may be removed from one bioreactor assembly and later added to another bioreactor assembly.

Any suitable bioreactor assembly may be used in the methods of the invention. The bioreactor assembly used in the methods of the invention can be the bioreactor assembly used for the bioconversion of syngas, or the bioreactor assembly used in the methods of the invention can be separate from the bioreactor assembly used for the bioconversion of syngas. The bioreactor assemblies for use in the methods of the invention include, but are not limited to, column reactors; bubble columns; jet loop reactors; stirred tank reactors; fluidized bed reactors; trickle bed reactors; biofilm reactors, including, but not limited to membrane bioreactors; and static mixer reactors including, but not limited to, pipe reactors. One or more bioreactors may be used, and when two or more bioreactors are used, they may be in parallel or sequential operation. The bioreactor assembly can, but is not always required to, include heat exchangers; solids separation unit operations such as centrifuges, settling ponds, and filters; gas/liquid separation unit operations; pumps; and equipment useful for monitoring and control of the bioreactor assembly.

A separate bioreactor assembly can, if desired, be integrated into the facility for the bioconversion of syngas to oxygenated organic compound. For instance, where the facility contains a distillation assembly, the distillation assembly may be used to remove at least a portion of the oxygenated product and to denature the aqueous fermentation broth.

As used herein, “biomass” refers to living or previously living (e.g., recently living) biological material, including plants and animals, and contains at least hydrogen, oxygen, and carbon. Biomass typically also contains nitrogen, phosphorus, sulfur, sodium, potassium, and trace metals. The chemical composition of biomass can vary from source to source and even within a source. Sources of biomass include, but are not limited to, harvested plants such as wood, grass clippings, and yard waste, switchgrass, corn (including corn stover), hemp, sorghum, sugarcane (including bagas), and the like, and waste such as garbage and municipal solid waste. Biomass does not include fossil fuels such as coal, natural gas, and petroleum.

Fossil carbonaceous materials, or fossil fuels, include, but are not limited to, natural gas; petroleum including carbonaceous streams from the refining or other processing of petroleum including, but not limited to, petroleum coke, lignite, and coal.

As used herein, “aqueous broth,” or “aqueous fermentation broth,” refers to a liquid water phase which may contain dissolved compounds including, but not limited to hydrogen, carbon monoxide, and carbon dioxide.

Intermittently means from time to time and may be at regular or irregular time intervals.

Syngas means a gas, regardless of source, containing at least one of hydrogen and carbon monoxide and may, and usually does, contain carbon dioxide.

Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas. Syngas is typically produced by a gasifier or reformer (steam, autothermal, or partial oxidation). Any of the aforementioned biomass sources are suitable for producing syngas. The syngas produced thereby will typically contain from about 10 to about 60 mole % CO, at least about 1 mole % CO₂, and preferably between about 35 and about 65 mole % H₂. The syngas may also contain N₂ and CH₄ as well as trace components such as H₂S, COS, NH₃, and HCN. Other sources of the gas substrate include gases generated during petroleum and petrochemical processing and from industrial processes. These gases may have substantially different compositions than typical syngas and may be essentially pure hydrogen or essentially pure carbon monoxide. The gas substrate may be obtained directly from gasification or from petroleum and petrochemical processing or industrial processes or may be obtained by blending two or more streams. Also, the gas substrate may be treated to remove or alter the composition including, but not limited to, removing components by chemical or physical sorption, membrane separation, and selective reaction.

The product oxygenated organic compounds produced in the methods of this invention will depend upon the organism or combination of organisms used for the fermentation and the conditions of the fermentation.

Any suitable organism (e.g., a non-naturally occurring organism) that contains a gene encoding an enzyme belonging to EC 1.2.3.1 may be utilized in the methods of this invention. For example, the non-naturally occurring organism utilized in the methods of this invention may comprise a sequence with at least about 95%, about 96%, about 97%, about 98%, about 99%, or about 100% identity to any one of SEQ ID NOs: 1-7.

Any suitable organism (e.g., a non-naturally occurring organism) that contains an exogenous gene encoding an enzyme belonging to EC 1.2.3.1 may be utilized in the methods of this invention. For example, the non-naturally occurring organism utilized in the methods of this invention may comprise a sequence with at least about 95%, about 96%, about 97%, about 98%, about 99%, or about 100% identity to any one of SEQ ID NOs: 1-7.

The aqueous broth is maintained under anaerobic fermentation conditions including a suitable temperature, for example, between about 25° C. and about 60° C., or in the range of about 30° C. to about 40° C. The conditions of fermentation, including the density of organisms and aqueous fermentation broth composition are preferably sufficient to achieve the sought conversion efficiency of hydrogen and carbon monoxide. The pH of the aqueous broth is acidic, for example, between about 4 and about 6.5.

The rate of supply of the syngas under steady state conditions to a bioreactor is preferably such that the rate of transfer of carbon monoxide and hydrogen to the liquid phase matches the rate that carbon monoxide and hydrogen are bioconverted. The rate at which carbon monoxide and hydrogen can be consumed will be affected by the nature of the organism, the concentration of the organism in the aqueous fermentation broth and the fermentation conditions. As the rate of transfer of carbon monoxide and hydrogen to the aqueous fermentation broth is a parameter for operation, conditions affecting the rate of transfer such as interfacial surface area between the gas and liquid phases and driving forces are important. Preferably the feed gas is introduced into the bioreactor in the form of microbubbles. Often the microbubbles have diameters in the range of about 0.01 to about 0.5 millimeter, or about 0.02 to about 0.3 millimeter.

In another aspect, the disclosure provides a method of preparing animal feed. As used herein, animal feed can be any suitable type of animal feed, such as, for example, aquatic culture (fish feed), poultry feed, cattle feed, hog feed, bird feed, etc. In an aspect, the removed acetogenic carboxydotrophic bacterium fraction is effective for use as animal feed. In a further aspect, the disclosure provides methods of preparing animal feed, the methods comprising: providing to a bioreactor: (1) a gaseous substrate comprising CO, CO₂, and optionally H₂, (2) at least one organism of an aspect of the present invention, and (3) a liquid nutrient medium, and providing conditions within the bioreactor for the at least one organism to convert CO, CO₂, and optionally H₂ to at least one oxygenated product, wherein the conditions within the bioreactor create a bioreactor broth; removing the bioreactor broth from the bioreactor to produce a removed broth, removing the at least one oxygenated product from the removed broth to produce an oxygenated product-depleted removed broth, and removing the at least one organism from the removed broth and/or the oxygenated product-depleted removed broth to produce a removed acetogenic carboxydotrophic bacterium fraction, wherein the removed acetogenic carboxydotrophic bacterium fraction is effective for use as animal feed. In an aspect, the oxygenated product-depleted removed broth and/or removed broth is provided to the bioreactor.

In some embodiments, the method of preparing animal feed is useful for producing aquatic culture containing relatively low amounts of one or more toxic metals, such as mercury, iron, nickel, etc.

In another aspect, the disclosure provides a method of preparing fertilizer. In an aspect, the removed acetogenic carboxydotrophic bacterium fraction is effective for use as fertilizer. In a further aspect, the disclosure provides methods of preparing fertilizer, the methods comprising: providing to a bioreactor: (1) a gaseous substrate comprising CO, CO₂, and optionally H₂, (2) at least one organism of an aspect of the present invention, and (3) a liquid nutrient medium, and providing conditions within the bioreactor for the at least one organism to convert CO, CO₂, and optionally H₂ to at least one oxygenated product, wherein the conditions within the bioreactor create a bioreactor broth; removing the bioreactor broth from the bioreactor to produce a removed broth, removing the at least one oxygenated product from the removed broth to produce an oxygenated product-depleted removed broth, and removing the at least one organism from the removed broth and/or the oxygenated product-depleted removed broth to produce a removed acetogenic carboxydotrophic bacterium fraction, wherein the removed acetogenic carboxydotrophic bacterium fraction is effective for use as fertilizer. In an aspect, the oxygenated product-depleted removed broth and/or removed broth is provided to the bioreactor.

In an aspect, the removed acetogenic carboxydotrophic bacterium fraction is effective for landfill application or land application as fertilizer or animal feed. In an aspect, the removed acetogenic carboxydotrophic bacterium fraction is useful for fertilizer, animal feed, landfill application, and/or land application contain protein, fat, carbohydrate, and/or minerals, e.g., 86% protein, 2% fat, 2% minerals, 10% carbohydrate. The removed acetogenic carboxydotrophic bacterium fraction can be effective for landfill application (e.g., to Class A solids), land application as fertilizer, or feed for animals, such as aquatic culture (fish feed), poultry feed, cattle feed, hog feed, etc. In the case of fish feed, advantageously, in some aspects, the fish feed contains less total toxic metals as compared with conventional fish meal.

In some aspects, the removed acetogenic carboxydotrophic bacterium fraction contains about 25 to about 50 wt. % solids, such as about 25 to about 40 wt. % solids. The amount of recovered solids is advantageous because it contains protein, carbohydrates, minerals, and potentially vitamins of nutritional value for plants and animals.

The removed acetogenic carboxydotrophic bacterium fraction can be used wet or dry. For example, in some aspects, the removed acetogenic carboxydotrophic bacterium fraction is effective for use as a wet fertilizer. In some aspects, the method further comprises drying the removed acetogenic carboxydotrophic bacterium fraction to form a “dried cake”, and the resulting dried cake is effective for use as dry fertilizer, animal feed or fish feed, or any combination thereof. The drying of the removed acetogenic carboxydotrophic bacterium fraction can also be used as a means to concentrate the removed acetogenic carboxydotrophic bacterium fraction. However, the removed acetogenic carboxydotrophic bacterium fraction can be dried using any suitable means. Drying may also be used as a means to preserve and/or stabilize the fraction to prevent microbial degradation or spoilage.

The respective compositions of the animal feed and fertilizer are generally similar because they are mainly composed of microbial proteins and/or carbohydrates. In some embodiments, the animal feed and/or fertilizer contains protein (e.g. from about 30 wt. % to about 90 wt. %, such as from about 60 wt. % to about 90 wt. %), fat (e.g. from about 1 wt. % to about 12 wt. %, such as from about 1 wt. % to about 3 wt. %), carbohydrate (e.g. from about 5 wt. % to about 60 wt. %, such as from about 15 wt. % to about 60 wt. %, or from about 5 wt. % to about 15 wt. %) and/or minerals such as sodium, potassium, copper etc. (e.g. from about 1 wt. % to about 20 wt. %, such as from about 1 wt. % to about 3 wt. %). For example, the animal feed and/or fertilizer can contain about 86% protein, about 2% fat, about 2% minerals, and about 10% carbohydrate. Aspects, including embodiments, of the subject matter described herein may be beneficial alone or in combination, with one or more other aspects or embodiments.

Without limiting the foregoing description, certain non-limiting aspects of the disclosure numbered 1-27 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) An organism comprising a highly expressed form of EC 1.2.3.1.

(2) The organism of aspect 1, wherein the enzyme belonging to EC 1.2.3.1 is aldehyde oxidoreductase (AOR).

(3) The organism of aspect 1 or 2, wherein the organism is a bacterium.

(4) The organism of aspect 1 or 2, wherein the organism is an acetogenic carboxydotrophic bacterium.

(5) The organism of aspect 1 or 2, wherein the organism is a Clostridium bacterium.

(6) The organism of any one of the aspects 1-5, wherein the organism further comprises a sequence with at least 95% identity to SEQ ID NO: 7.

(7) A method for efficient fermentation broth recycle, the method comprising:
(a) providing to a bioreactor: (1) a gaseous substrate comprising CO, CO₂, and optionally H₂, (2) at least one organism of any one of aspects 1-6, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

(8) A method for improving bottoms recycle, the method comprising:
(a) providing to a bioreactor: (1) a gaseous substrate comprising CO, CO₂, and optionally H₂, (2) at least one organism of any one of aspects 1-6, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

(9) A method for converting CO, CO₂, and optionally H₂ to ethanol, the method comprising:
(a) providing to a bioreactor: (1) a gaseous substrate comprising CO, CO₂, and optionally H₂, (2) at least one organism of any one of aspects 1-6, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

(10) The method of any one of aspects 7-9, wherein the oxygenated product is ethanol.

(11) The method of any one of aspects 7-9, wherein the oxygenated product is acetic acid, butyrate, butanol, propionate, or propanol.

(12) The method of any one of aspects 7-11, wherein the exogenous gene encoding an enzyme belonging to EC 1.2.3.1 in the at least one organism is expressed at least about 2 times as compared to an organism that is the same except for not having an exogenous gene encoding an enzyme belonging to EC 1.2.3.1 when the at least one organism and the organism that does not have an exogenous gene encoding an enzyme belonging to EC 1.2.3.1 are cultured under similar culturing conditions.

(13) The method of any one of aspects 7-12, wherein at least about 5% more of the at least one oxygenated product is produced by the organism as compared to an organism that is the same except for not having an exogenous gene encoding an enzyme belonging to EC 1.2.3.1 when the at least one organism and the organism that does not have an exogenous gene encoding an enzyme belonging to EC 1.2.3.1 are cultured under similar culturing conditions.

(14) The method of any one of aspects 7-13, wherein the at least one organism is an acetogenic carboxydotrophic bacterium.

(15) The method of aspect 14, wherein the acetogenic carboxydotrophic bacterium is cultured in the bioreactor to produce an acetogenic carboxydotrophic bacterium culture.

(16) The method of aspect 15, wherein the acetogenic carboxydotrophic bacterium culture continuously produces at least one oxygenated product for more than about 24 hours.

(17) The method any one of aspects 7-16, further comprising:
(c) removing the bioreactor broth from the bioreactor to produce a removed broth,
(d) removing the at least one oxygenated product from the removed broth to produce an oxygenated product-depleted removed broth,
   (e) removing the at least one organism from the removed broth and/or the oxygenated product-depleted removed broth, and
   (f) providing the oxygenated product-depleted removed broth to the bioreactor.

(18) The method of aspect 17, wherein at least about 1 gram of the at least one oxygenated product is produced per liter of removed broth.

(19) The method of aspect 17 or 18, wherein more than about 80% of the oxygenated product-depleted removed broth is provided to the bioreactor.

(20) The method of aspect 14 or 15, wherein the acetogenic carboxydotrophic bacterium culture is removed from the bioreactor to produce a removed acetogenic carboxydotrophic bacterium fraction.

(21) The method of aspect 19, wherein the removed acetogenic carboxydotrophic bacterium fraction is concentrated.

(22) The method of aspect 19 or 20, wherein the removed acetogenic carboxydotrophic bacterium fraction is concentrated by drying.

(23) The method of any one of aspects 19-22, wherein the removed acetogenic carboxydotrophic bacterium fraction is effective for use as animal feed.

(24) The method of any one of aspects 19-22, wherein the removed acetogenic carboxydotrophic bacterium fraction is effective for use as fertilizer.

(25) A method of preparing animal feed, the method comprising:
(a) providing to a bioreactor: (1) a gaseous substrate comprising CO, CO₂, and optionally H₂, (2) at least one organism of any one of aspects 1-6, and (3) a liquid nutrient medium, and
(b) providing conditions within the bioreactor for the at least one organism to convert CO, CO₂, and optionally H₂ to at least one oxygenated product, wherein the conditions within the bioreactor create a bioreactor broth;
(c) removing the bioreactor broth from the bioreactor to produce a removed broth,
(d) removing the at least one oxygenated product from the removed broth to produce an oxygenated product-depleted removed broth, and
(e) removing the at least one organism from the removed broth and/or the oxygenated product-depleted removed broth to produce a removed acetogenic carboxydotrophic bacterium fraction,
wherein the removed acetogenic carboxydotrophic bacterium fraction is effective for use as animal feed.

(26) A method of preparing fertilizer, the method comprising:
(a) providing to a bioreactor: (1) a gaseous substrate comprising CO, CO₂, and optionally H₂, (2) at least one organism of any one of aspects 1-6, and (3) a liquid nutrient medium, and
(b) providing conditions within the bioreactor for the at least one organism to convert CO, CO₂, and optionally H₂ to at least one oxygenated product, wherein the conditions within the bioreactor create a bioreactor broth;
(c) removing the bioreactor broth from the bioreactor to produce a removed broth,
(d) removing the at least one oxygenated product from the removed broth to produce an oxygenated product-depleted removed broth, and
(e) removing the at least one organism from the removed broth and/or the oxygenated product-depleted removed broth to produce a removed acetogenic carboxydotrophic bacterium fraction,
wherein the removed acetogenic carboxydotrophic bacterium fraction is effective for use as fertilizer.

(27) The method of aspect 25 or 26, further comprising providing the oxygenated product-depleted removed broth to the bioreactor.

The following examples further illustrate the invention but, of course, should not be construed as in any way limiting its scope.

### Example 1

This example demonstrates that the amount of non-W AOR gene expression in SB1 is different than that of C. autoethanogenum.

Organisms SB1 and C. autoethanogenum were cultured under steady state conditions and then analyzed using RNA seq analysis to determine the level of expression of some genes necessary for production of ethanol.

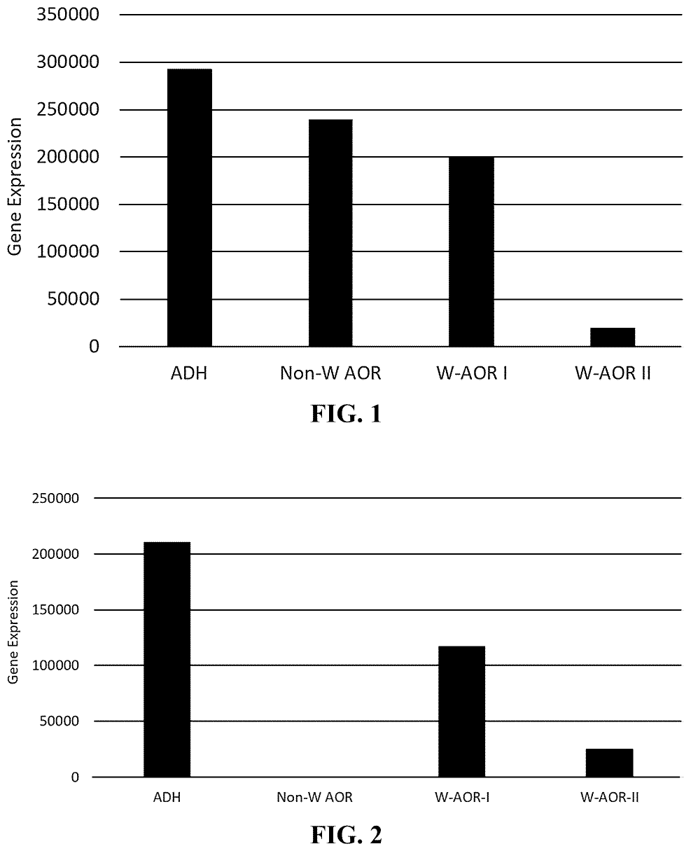

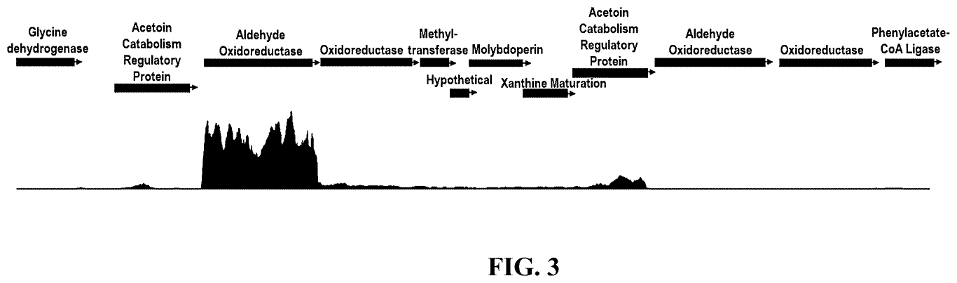

FIGS. 1 and 2 are graphs showing the average amount of gene expression for the indicated genes in organisms SB1 and C. autoethanogenum, respectively. The gene expression derived from RNA seq data for each organism was plotted. As is apparent from the data presented in FIGS. 1 and 2, the average amount of gene expression for the indicated genes is different between the organisms. Although a small amount of expression of EC 1.2.3.1 (Non-W AOR) was detectable in C. autoethanogenum, it cannot be seen in FIG. 2.

### Example 2

This example describes the differing genome architectures surrounding the non-W AOR genes (EC 1.2.3.1) in organisms SB1 and C. autoethanogenum, as well as differences in expression.

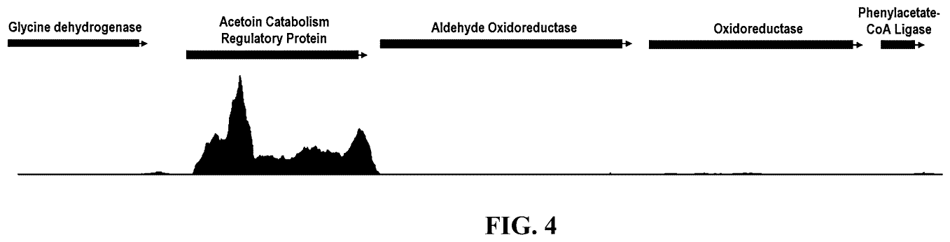

FIG. 3 shows the genome architecture in SB1, which shows the presence of two copies of the EC 1.2.3.1 class enzyme, with an intervening region composed of a methyltransferase, hypothetical protein, and molybodterin cofactor biosynthesis protein. In both cases the EC 1.2.3.1 enzyme is directly flanked by a predicted acetoin catabolism regulatory protein and a predicted oxidoreductase. The lower part of FIG. 3 shows the read density in SB1. SB1 and C. autoethanogenum were cultured under identical steady state conditions and then analyzed using RNA seq analysis to determine the level of expression of nine genes. The read density derived from RNA seq data (which is proportional to the level of gene expression) for each organism was plotted. Higher values on the plots are proportional to higher levels of gene expression. The upstream non-W aldehyde oxidoreductase (EC 1.2.3.1) gene shows a higher read density, whereas the gene corresponding to the downstream EC 1.2.3.1 shows a lower read density.

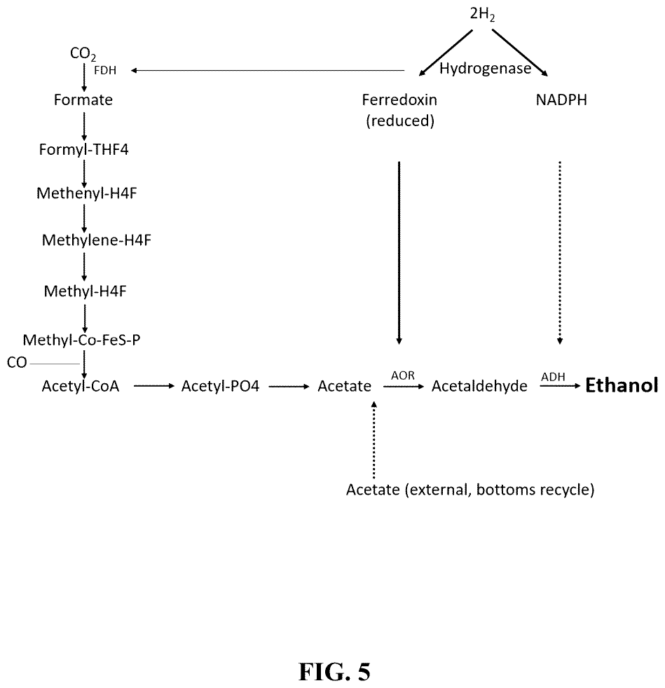

FIG. 4 shows the corresponding region surrounding the non-W aldehyde oxidoreductase gene in C. autoethanogenum. Unlike SB1, only a single copy of the gene encoding the EC 1.2.3.1 enzyme is present. Furthermore read density plots show the gene to be expressed at a low level.

### Example 3

This example shows the importance of the non-tungsten AOR gene on strain performance.

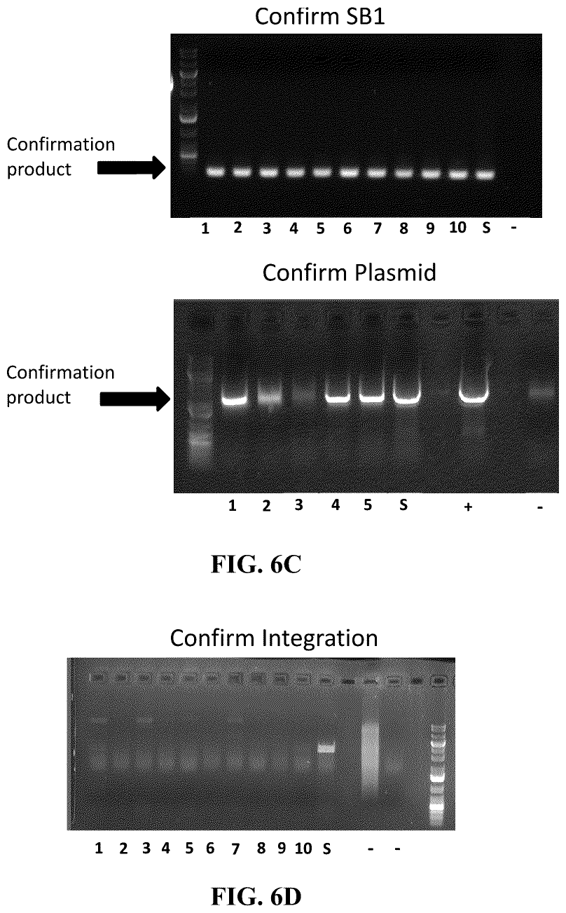

An effort was made to disrupt the non-tungsten AOR gene using a single crossover method (for a detailed description of the method, see U.S. Provisional Patent Application 63/369,683). A fragment was amplified via PCR that contained a 1978 bp fragment spanning the region from nucleotide 400 to nucleotide 2378 of SEQ ID NO: 6. The resulting fragment would encode a form of the non-tungsten AOR that lacks 400 bp at the 5′ and 3′ regions. The fragment was cloned into a derivative of plasmid pMTL84151 (Heap, et al., Journal of Microbiological Methods, 78(1): 79-85 (2009)) to form plasmid pKO32 (FIG. 6A). Upon single cross-over recombination between the plasmid and the corresponding genes on the chromosome, the plasmid would be expected to integrate into the chromosome leading to disruption of the highly expressed non-tungsten AOR gene.

Plasmid pKO32 was introduced via conjugation and single crossover integrations were selected using 2 ug/ml thiamphenicol to select for plasmid pKO32 and 10 ug/ml trimethoprim to counterselect against the E. coli donor strain. After conjugation, multiple small antibiotic resistant colonies were isolated (FIG. 6B). Ten colonies (labeled 1-10), and a streak of multiple colonies (labeled S) were confirmed to be strain SB1 by PCR and gel electrophoresis (FIG. 6C, top panel). Five of these confirmed SB1 colonies (labeled 1-5), and a streak of a multiple colonies (labeled S) were then screened in a similar manner for presence of the plasmid (FIG. 6C, bottom panel). Colonies that were confirmed to be SB1 and to contain the plasmid were then tested for integration of the plasmid into the non-tungsten AOR gene using PCR (FIG. 6D). The small antibiotic resistant SB1 colonies identified were shown to contain the plasmid (FIG. 6C), but the plasmid was not integrated into the genome (FIG. 6D). Similar types of small colonies have been observed by other groups (Minton, et al., Anaerobe, 41: 104-112 (2016)), and have been suggested to result from low-level replication of pMTL84151 in Clostridial strains. The identification of the plasmid within SB1 and the lack of confirmed integration events strongly suggests that integration of the plasmid (and corresponding disruption of the gene encoding the non-tungsten AOR) leads to a lack of cell viability. Consistent with this, additional attempts to disrupt the gene using similar strategies led to periodic recovery of confirmed integration events (data not shown). However, these integration events were associated with duplications that retained an intact copy of the non-tungsten AOR remaining in the genome.

Disruptions of the non-tungsten AOR could not be isolated, likely because disruption of the gene led to a non-viable cell. These results indicate the importance of the non-tungsten AOR for the growth of SB1.

### Example 4

This example shows the effect of expression of the non-tungsten AOR on performance of C. autoethanogenum during recycle.

The region of the SB1 genome corresponding to the non-tungsten AOR and upstream regulatory sequences (SEQ ID NO: 7) are cloned into pMTL83151 (Heap, et al., Journal of Microbiological Methods, 78(1): 79-85 (2009)), and introduced into C. autoethanogenum by conjugation. Exconjugants containing the plasmid and SEQ ID NO: 7 are isolated using 2 ug/ml thiamphenicol to select for plasmid and 10 ug/ml trimethoprim to counterselect against the E. coli donor strain. The confirmed plasmid-containing strains are then subjected to fermentation in the presence of 2 ug/ml thiamphenicol during 88% bottoms recycle using conditions previously described (see U.S. Provisional Patent Application 63/298,426). C. autoethanogenum containing plasmid-borne SEQ ID NO: 7 maintained a higher productivity during growth in 88% bottoms recycle than did C. autoethanogenum lacking SEQ ID NO: 7.

These results indicate that SEQ ID NO: 7 (containing the non-tungsten AOR) leads to improved performance during growth in 88% bottoms recycle.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An organism comprising a highly expressed form of EC 1.2.3.1.

2. The organism of claim 1, wherein the enzyme belonging to EC 1.2.3.1 is aldehyde oxidoreductase (AOR).

3. The organism of claim 1, wherein the organism is a bacterium.

4. The organism of claim 1, wherein the organism is an acetogenic carboxydotrophic bacterium.

5. The organism of claim 1, wherein the organism is a Clostridium bacterium.

6. The organism of claim 1, wherein the organism further comprises a sequence with at least 95% identity to SEQ ID NO: 7.

7. A method for efficient fermentation broth recycle, the method comprising:
(a) providing to a bioreactor: (1) a gaseous substrate comprising CO, CO₂, and optionally H₂, (2) at least one organism of claim 1, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

8. A method for improving bottoms recycle, the method comprising:
(a) providing to a bioreactor: (1) a gaseous substrate comprising CO, CO₂, and optionally H₂, (2) at least one organism of claim 1, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

9. A method for converting CO, CO₂, and optionally H₂ to ethanol, the method comprising:
(a) providing to a bioreactor: (1) a gaseous substrate comprising CO, CO₂, and optionally H₂, (2) at least one organism of claim 1, and (3) a liquid nutrient medium, and
(b) providing conditions within the bioreactor for the at least one organism to convert CO, CO₂, and optionally H₂ to at least one oxygenated product,
wherein the conditions within the bioreactor create a bioreactor broth.

10. The method of claim 7, wherein the oxygenated product is ethanol.

11. The method of claim 7, wherein the oxygenated product is acetic acid, butyrate, butanol, propionate, or propanol.

12. The method of claim 7, wherein the exogenous gene encoding an enzyme belonging to EC 1.2.3.1 in the at least one organism is expressed at least about 2 times higher as compared to an organism that is the same except for not having an exogenous gene encoding an enzyme belonging to EC 1.2.3.1 when the at least one organism and the organism that does not have an exogenous gene encoding an enzyme belonging to EC 1.2.3.1 are cultured under similar culturing conditions.

13. The method of claim 7, wherein at least about 5% more of the at least one oxygenated product is produced by the organism as compared to an organism that is the same except for not having an exogenous gene encoding an enzyme belonging to EC 1.2.3.1 when the at least one organism and the organism that does not have an exogenous gene encoding an enzyme belonging to EC 1.2.3.1 are cultured under similar culturing conditions.

14. The method of claim 7, wherein the at least one organism is an acetogenic carboxydotrophic bacterium.

15. The method of claim 14, wherein the acetogenic carboxydotrophic bacterium is cultured in the bioreactor to produce an acetogenic carboxydotrophic bacterium culture.

16. The method of claim 15, wherein the acetogenic carboxydotrophic bacterium culture continuously produces at least one oxygenated product for more than about 24 hours.

17. The method of claim 7, further comprising:
(c) removing the bioreactor broth from the bioreactor to produce a removed broth,
(d) removing the at least one oxygenated product from the removed broth to produce an oxygenated product-depleted removed broth,
(e) removing the at least one organism from the removed broth and/or the oxygenated product-depleted removed broth, and
(f) providing the oxygenated product-depleted removed broth to the bioreactor.

18. The method of claim 17, wherein at least about 1 gram of the at least one oxygenated product is produced per liter of removed broth.

19.-24. (canceled)

25. A method of preparing animal feed, the method comprising:
(a) providing to a bioreactor: (1) a gaseous substrate comprising CO, CO₂, and optionally H₂, (2) at least one organism of claim 1, and (3) a liquid nutrient medium, and
(b) providing conditions within the bioreactor for the at least one organism to convert CO, CO₂, and optionally H₂ to at least one oxygenated product, wherein the conditions within the bioreactor create a bioreactor broth;
(c) removing the bioreactor broth from the bioreactor to produce a removed broth,
(d) removing the at least one oxygenated product from the removed broth to produce an oxygenated product-depleted removed broth, and
(e) removing the at least one organism from the removed broth and/or the oxygenated product-depleted removed broth to produce a removed acetogenic carboxydotrophic bacterium fraction,
wherein the removed acetogenic carboxydotrophic bacterium fraction is effective for use as animal feed.

26. A method of preparing fertilizer, the method comprising:
(a) providing to a bioreactor: (1) a gaseous substrate comprising CO, CO₂, and optionally H₂, (2) at least one organism of claim 1, and (3) a liquid nutrient medium, and
(b) providing conditions within the bioreactor for the at least one organism to convert CO, CO₂, and optionally H₂ to at least one oxygenated product, wherein the conditions within the bioreactor create a bioreactor broth;
(c) removing the bioreactor broth from the bioreactor to produce a removed broth,
(d) removing the at least one oxygenated product from the removed broth to produce an oxygenated product-depleted removed broth, and
(e) removing the at least one organism from the removed broth and/or the oxygenated product-depleted removed broth to produce a removed acetogenic carboxydotrophic bacterium fraction,
wherein the removed acetogenic carboxydotrophic bacterium fraction is effective for use as fertilizer.

27. (canceled)
